(19) United States
(12) Patent Application Publication
(10) Pub. No.: US 2026/0069894 A1
(43) Pub. Date: Mar. 12, 2026

(54) Ultrasound Therapy System Guided by Three-Dimensional Ultrasound Images

(71) Applicant: Precision Target Ultrasound, Inc., Redmond, WA (US)

(72) Inventors: Xiaocong GUO, Woodinville, WA (US); Zhenyu CHEN, Woodinville, WA (US)

(73) Assignee: Precision Target Ultrasound, Inc., Redmond, WA (US)

(21) Appl. No.: 19/305,832

(22) Filed: Aug. 21, 2025

Related U.S. Application Data
(60) Provisional application No. 63/693,539, filed on Sep. 11, 2024.

(51) Int. Cl. A61N 7/00; B06B 1/06

(52) U.S. Cl. CPC A61N 7/00; B06B 1/0629; A61N 2007/0082; A61N 2007/0095; B06B 2201/76

(57) ABSTRACT

An ultrasound therapy system has both a therapy transducer configured to transmit therapeutic ultrasound energy toward a target tissue and an imaging transducer configured to image the target tissue over a three-dimensional volume. The transducers are preferably arranged in a common housing but have respective controllers. The therapy controller causes the therapy transducer to focus the therapeutic ultrasound energy at a selectable focal position within a three-dimensional treatment region and the imaging controller generates a three-dimensional image of the target tissue, which is displayed for viewing by a user. Target location information is then transferred from the imaging controller to the therapy controller. The therapy controller adjusts the focal position of the therapeutic ultrasound energy within the three-dimensional treatment region accordingly.

### REFERENCE TO RELATED APPLICATIONS

This application claims priority of U.S. Provisional Ser. No. 63/693,539 , filed 11 Sep. 2024.

### TECHNICAL FIELD

This disclosure relates generally to systems for therapeutic use of ultrasound.

### BACKGROUND ART

High Intensity Focused Ultrasound (HIFU) and Histotripsy are advanced therapeutic methods that use ultrasound technology for precise medical interventions.

Whereas HIFU employs high-energy ultrasound to activate or destroy targeted tissue, histotripsy involves the mechanical disruption of tissue through the generation of controlled cavitation bubbles. This method, unlike HIFU, doesn't rely on thermal effects but instead uses rapid pressure changes to liquefy targeted tissue. To achieve effective histotripsy, it's essential to ensure sufficiently high acoustic intensity. In tumor therapy, ultrasound with a center frequency ranging from 750 kHz to 1 MHz is typically employed, with peak negative pressure levels between 10 and 27.9 MPa. Positive pressure readings often exceed 80 MPa, correlating with a negative pressure of 15 MPa (see, for example, Eli Vlaisavljevich, et al., “Histotripsy-induced cavitation cloud initiation thresholds in tissues of different mechanical properties”, IEEE Trans Ultrason. Ferroelectr. Freq. Control, 61(2): 341-352February 2014).

In both HIFU and Histotripsy, during the treatment phase, ultrasound energy is emitted from a transducer when subjected to high voltage. To achieve the required intensity, the ultrasound beam is carefully controlled to focus on a specific point. This focusing is typically achieved through physical mechanisms rather than electrical methods or phase control. A typical physical focusing transducer that uses a single piezoelectric element is illustrated in FIGS. 1A and 1B, and a conventional transducer that has a few elements, or a composition of numerous elements with larger dimensions much larger (at least two times as large) than the wavelength (center frequency) of the emitted wave is illustrated in FIGS. 2A and 2B.

Physical focusing is simpler than electrical focusing, but it comes with the limitation that the focus point cannot be moved unless the transducer is physically adjusted. In cases where the transducer remains stationary, the intensity diminishes significantly outside the focal point. Mechanical movement of the focus point is therefore necessary, whether at the onset of treatment or when treating different positions of the target tissue.

In contrast, electrical focusing or phase control allows for changing the focus point while keeping the transducer in a fixed position. This is particularly advantageous when the energy intensity is sufficiently high within a specific range. Operating with this method is more convenient as the transducer only needs to be placed near the target tissue, with the focus position adjusted by altering the phase of the ultrasound energy transmitted through the transducer elements.

Of course, before attempting to destroy target tissue such as a tumor, one must know where it is so as to adjust the transducer's point of focus accordingly. Imaging using ultrasound is typically used to guide the operator. In such a guidance phase, two-dimensional ultrasound images are generally used. As is known, these two-dimensional images are an average representation from the thickness direction. This may, however, lack precision, especially when employed for treatment guidance.

### BRIEF DESCRIPTION OF THE DRAWINGS

FIGS. 1A and 1B illustrated a single-element, physical focus transducer commonly used in the prior art.

FIGS. 2A and 2B depict a multi-element physical focus transducer according to the prior art.

FIG. 3 shows the main components of a system architecture used in the embodiments of the invention.

FIG. 4 illustrates the basic electrical and driving circuitry of a group of ultrasound elements.

FIG. 5 depicts a therapeutic ultrasound transducer with matrix control using beamforming.

FIGS. 6 and 7 illustrate an ultrasound imaging transducer at the center of a therapy transducer.

FIGS. 8 and 9 illustrate an imaging transducer located off-center and at the inner edge of a therapy transducer.

FIGS. 10 and 11 illustrate a side-and off-center, corner-mounted imaging transducer, respectively.

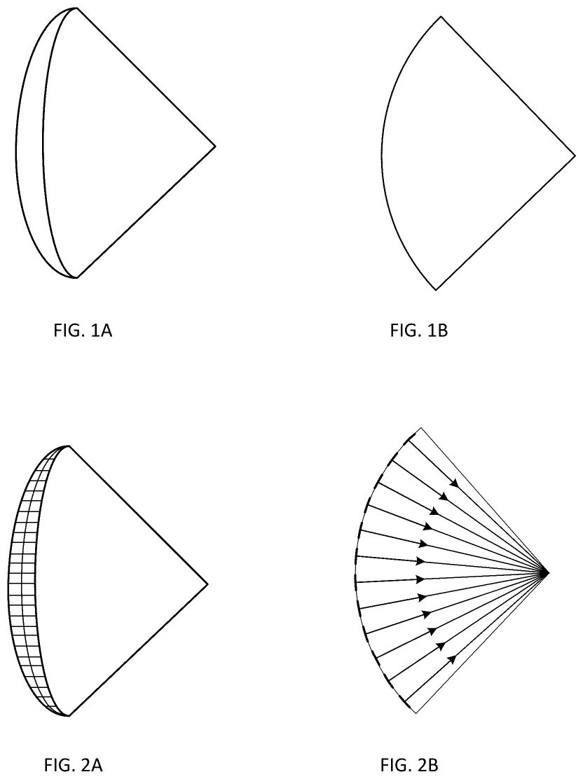

### DESCRIPTION OF THE INVENTION

This invention provides a new therapy system with dual matrix transducers that generate three-dimensional ultrasound images as a guide and transmit ultrasound energy three-dimensionally to destroy target tumor tissues to achieve therapeutic results.

At the highest level, the system comprises a treatment component and a guidance component that work seamlessly together, the guidance component being configured to share information with the treatment component. The invention provides two main configurations, of which there are different embodiments of each. In a “basic” embodiment, the imaging transducer, which may be one-dimensional, is a separate unit that is mechanically connected with the therapy transducer and is moved mechanically.

In a more flexible, “integrated” embodiment, the imaging array is substantially integrated with the therapy array in that the piezoelectric elements of both may be included in a single, common housing, even with array elements in the same plane, albeit separately energized and focused by respective controllers. This more flexible, integrated embodiment will be described first.

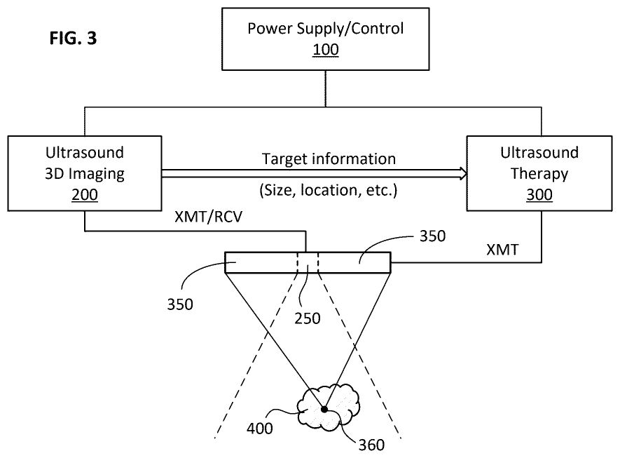

The general system architecture of the invention is illustrated in FIG. 3, which shows a power supply and related controller 100, which provides power to a 3D ultrasound imaging controller 200 and an ultrasound therapy controller 300, which are operatively connected using known circuitry to their respective transducers. The imaging controller 200 will include known hardware and software components such as a beamformer, etc., and the therapy controller 300 will include similar beamforming circuitry.

The system also includes one or more processors and an arrangement of volatile and/or non-volatile storage and memory, I/O and other conventional hardware and system software components to carry out the various calculations and processes needed for such procedures as beamforming, image composition and display, user interface control, etc. The different procedures described in this application may then be embodied as executable code stored in the storage and memory devices to direct the processor(s) to carry out the required operations. The controllers 200, 300 may include such processors themselves, or may use the processing capability of a central processor. These components and system configuration are not illustrated specifically in the figures because they are well known.

The imaging controller 200 generates appropriately phased transmit (XMT) signals to the elements of an array—a matrix—of piezoelectric elements forming a 2D array 250, and receives (RCV) the return signals that it then converts into data from which a 3D image can be formed for a user to view, using any conventional display arrangement. Note that even though the element matrix may be two-dimensional, signals for 3D images can be generated by configuring the controller 200 to apply known techniques to vary the point of focus throughout a 3D region.

The therapy controller 300 similarly applies transmit signals to an array of piezoelectric elements forming a 2D-matrix to form a 3D focus therapy transducer 350, and selectably steer the transducer's point of focus 360 into target tissue 400, such as a tumor. The imaging controller 200 provides target information such as location and size via any known communication link, such as over a data bus, writing to and reading from shared memory, etc., to the therapy controller 300, which can then direct the beam of the therapy transducer to focus on the tumor.

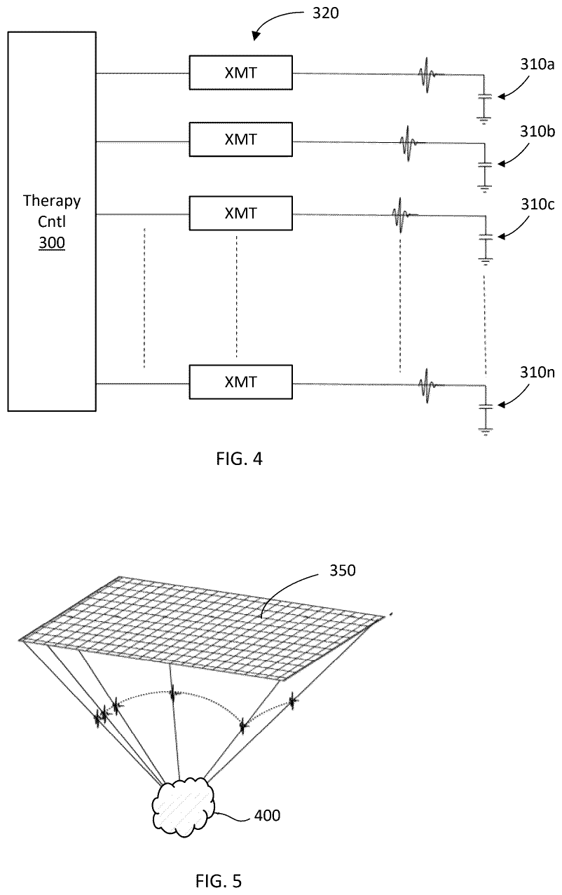

In the treatment component, ultrasound energy is thus emitted through the two-dimensional matrix ultrasound therapy transducer 350. As FIG. 4 shows, each element 310a, 310b, . . . , 310n within this matrix is linked to an individual transmit channel and transmitter (XMT, labelled collectively 320). The system then employs known beamforming techniques and circuitry to precisely control transmit starting time or delay for each element.

Using the beamforming mechanism, the energy from each element of the array 350 is controlled to focus on a predetermined position within the target tissue 400 in three dimensions. The focus position of the ultrasonic energy can be dynamically adjusted by manipulating the delay time. This capability allows for movement within a specified 3D region, which enables the comprehensive destruction of even medium-sized tumors, including those up to 5 cm in the liver, at standard depths.

To ensure optimal performance, the inventors established specific, preferred design criteria. The element pitch, or the distance between adjacent elements, should be less than 1.5 times the generated ultrasound wavelength at the center frequency of the transmitted ultrasound. This criterion is necessary for achieving precise focusing and heightened resolution during therapeutic interventions. Due to the small element dimension, the number of elements of the therapy array 350 will in many implementations be more than 1000 in order to achieve enough pressure; this is illustrated in FIG. 5 (albeit with fewer than 1000 elements shown, for clarity).

This system design eliminates the need for repositioning the ultrasonic transducer during surgery. The flexibility to alter the focus position within the designated range while maintaining a stable transducer position enhances the precision and efficiency of the therapeutic process. This technological advancement represents a significant breakthrough in the non-invasive treatment of tumors, particularly in scenarios involving complex anatomies or delicate surgical procedures.

In the imaging/guidance component 200/250, a three-dimensional image provides users, such as doctors, with a comprehensive view of the target tissue. These three-dimensional images can be generated in real-time or within a short timeframe, offering immediate and detailed visualization during medical intervention. Real-time, three-dimensional images in embodiments are generated using a sub-system that includes the two-dimensional matrix transducer 250, whose point of focus is dynamically controlled in three dimensions by adjusting the delay of array element transmissions. Following the completion of the transmission, each array element receives the reflected signals from the tissue. In other words, the imaging subsystem of embodiments of the invention operates like other ultrasound imaging systems, but with a novel difference, namely, the integration with a therapy subsystem so as to be able to provide guidance to a therapy transducer, also in real time.

To create a three-dimensional image, these reflected signals undergo conventional analog-to-digital conversion and are processed using any known beamforming technique. As is known, beamforming involves the combination and synchronization of these digital signals to construct a coherent and detailed three-dimensional representation of the target tissue.

The invention is thus able to provide real-time, high-resolution three-dimensional images during medical interventions. The ability to dynamically control the focused position and rapidly process reflected signals ensures accurate and up-to-date visualization, making available to medical professionals valuable information for precise decision-making and effective execution of therapeutic procedures.

In the basic embodiments of the invention, the rapid generation of a three-dimensional image is done using a system employing a mechanically movable one-dimensional ultrasonic transducer. Using known techniques, multiple two-dimensional images from diverse positions may be acquired using this transducer. The system then reconstructs these images into a cohesive three-dimensional representation based on the varying positions of the transducer.

The mechanical movement of the one-dimensional ultrasonic transducer allows for the acquisition of a series of two-dimensional images from different perspectives. These images are subsequently processed by the imaging transducer controller and combined to form a comprehensive and real-time three-dimensional image. The reconstruction process takes into account the specific positions of the transducer during image acquisition, ensuring accuracy and coherence in the final three-dimensional visualization.

This advanced imaging capability significantly enhances the user's ability to precisely identify and navigate the target tissue in three dimensions. The real-time or rapidly generated three-dimensional images provide crucial insights, allowing for accurate planning and execution of therapeutic interventions. This not only improves the efficiency of the procedure but also enhances the overall precision and success of the treatment.

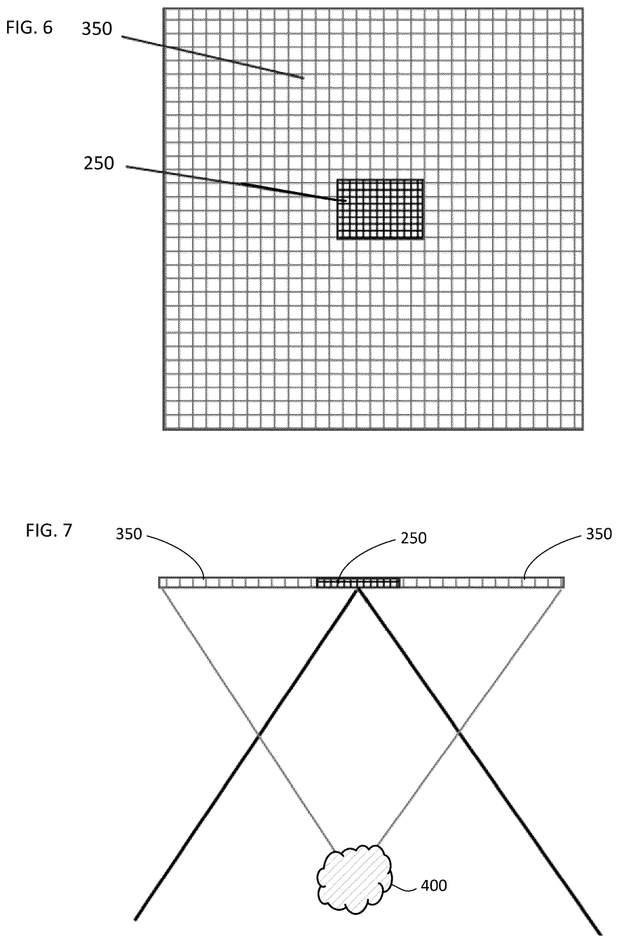

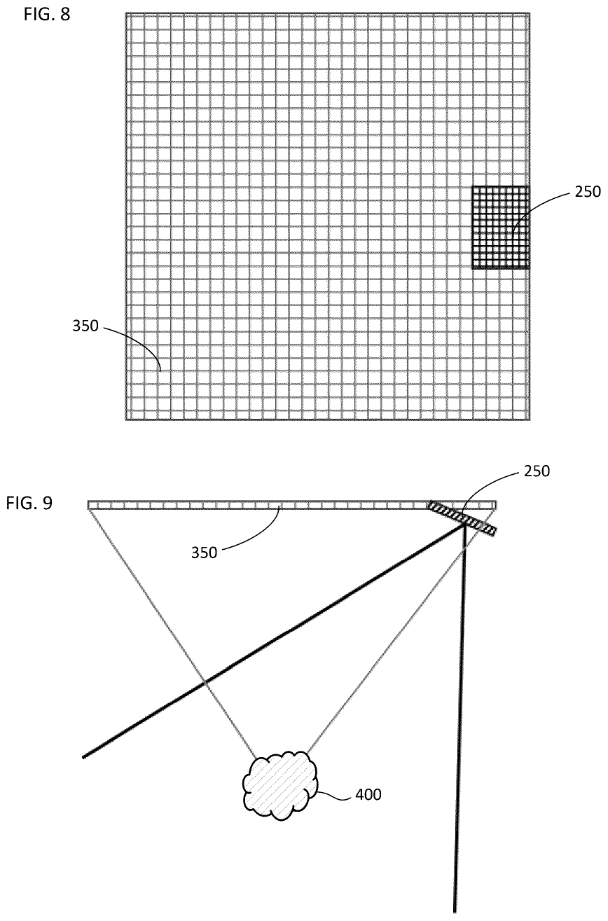

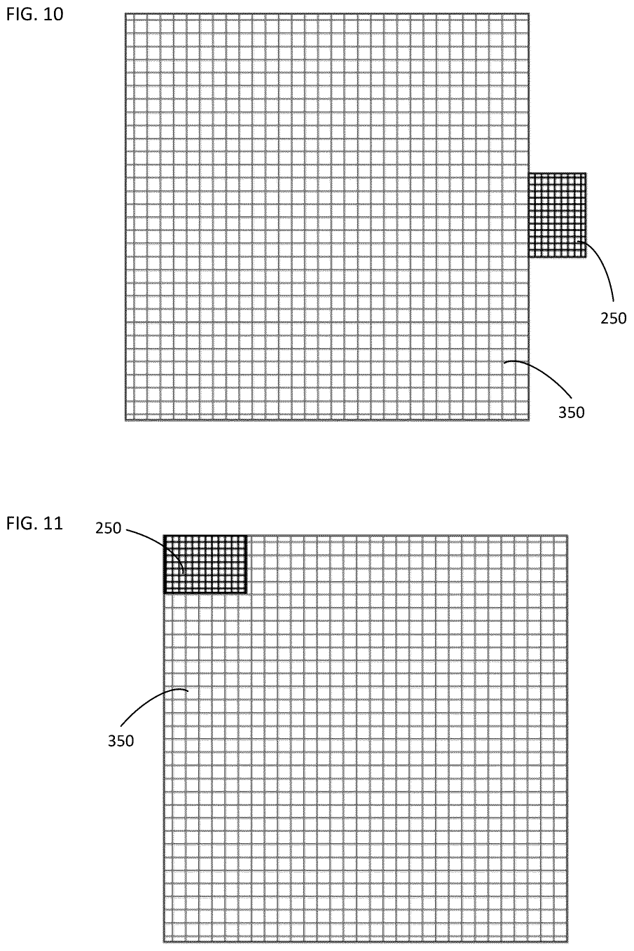

In a different embodiment, both the imaging and therapy transducers are formed as arrays of piezoelectric elements, with no need to mechanical steering rather, steering of the respective beams may be accomplished using known beam-steering techniques, involving the control of the timing and phasing of energizing of the various elements. The imaging transducer may take the form of a convex array, linear array, or phased array. The placement of the imagine array relative to the therapy array may be chosen depending on the preferences of the designer, for example, at the center (FIGS. 6 and 7), inner edge (FIGS. 8 and 9), outer edge (FIG. 10), or corner (FIG. 11). Note that when the imaging transducer 250 is not centrally located, as illustrated in FIGS. 6 and 7, there will be some difference in the central axes of two transducers, but these can still be oriented so as to ensure comprehensive coverage of the therapeutic region within the imaging field.

By integrating rapid three-dimensional imaging into the guidance part, the system ensures that medical professionals have access to up-to-date and detailed information, facilitating informed decision-making and optimizing the therapeutic outcomes in real-time scenarios.

At the initiation of treatment, the three-dimensional ultrasound image plays a pivotal role in identifying the target tissue, capturing crucial details such as its location, size, and shape. Before starting therapy, that is, before transmission of the therapeutic ultrasound beams into the patient, the user needs to identify the target. While viewing the 3D image of the general target region, the user may maneuver the imaging probe to capture a view of the target region. Using any known method, such as positioning a cursor, the user may then mark the target tumor in the image. Again using known image processing techniques, for example, edge detection, analysis of the difference in brightness, etc., the system may then identify the 3D boundaries of the tumor and “lock onto” it, even if it moves relative to the transducer, at least within some threshold range. Note that, since the image the user views is the result of 3D positional information (elevation, azimuth and depth) for each “pixel” of the image, marking and identifying the target location will also be able to provide the information about the geometry, such as current coordinates defining its position, shape and size, which the imaging controller then can pass to the therapy controller so that it can focus the therapeutic ultrasound beam onto the target.

This comprehensive information may thus be seamlessly transferred directly to the treatment part. Following user confirmation, ultrasound energy may then transmitted with precision to the identified target tissue.

As an alternative to automatic determination of target geometry after initial identification and marking by the user, the system could also enable real-time, user-directed “aiming”. In this embodiment, the therapy transducer controller could compute the point of focus of the therapeutic beam and transmit the coordinates of that point, relative to the transducer, to the imaging transducer controller. The imaging controller may then indicate to the user what the point of focus of the therapeutic beam would be if activated. The user may then maneuver the probe until the point of “aim” is within what he sees in the image is the tumor, and then activate the therapy probe to destroy that point. In short, in this embodiment, rather than the imaging transducer supplying the target coordinates to the therapy transducer, the therapy transducer may supply its instantaneous “point of aim” coordinates to the imaging controller, which then guides the user during real-time therapy.

Throughout treatment, real-time monitoring is, in all the embodiments, facilitated by the continuous use of ultrasound imaging. In the event of any movement of the target outside of a predetermined, acceptable range, the system may recognize the change and automatically halt treatment, that is, stop transmission of energy from the therapy transducer, issue a warning to the user, re-acquire and re-identify the target information, and await user confirmation. Once confirmed, the treatment may resume, ensuring not only accuracy but also safety during the therapeutic process.

This dynamic feedback mechanism, integrating real-time monitoring and adaptive response, underscores the system's ability to account for changes in the target's position. It not only improves the accuracy of the treatment but also prioritizes patient safety by providing timely warnings and requiring user confirmation before proceeding.

The new system provided by embodiments of the invention offers several significant benefits:

### Simplified Transducer Placement

The system reduces the position requirements for transducer placement, significantly decreasing the complexity and time involved in the operation.
There's minimal need to change the transducer's position during the entire treatment process, except in cases of exceptionally large tumors.
This ease-of-use contrasts favorably with geometric two-dimensional focus treatment systems, where fixed focusing requires mechanical adjustments, making the therapy operation more straightforward.

### Enhanced Accuracy with Three-Dimensional Image Guidance

The use of three-dimensional image guidance ensures accurate identification of the target, minimizing errors associated with two-dimensional images.
The precision provided by three-dimensional imaging improves the overall efficacy of the treatment, contributing to better outcomes.

### Adaptive Treatment Adjustments

The system's capability for three-dimensional movable treatment positions allows for convenient initiation and adjustments based on the evolving progress of the target.
Changes in target information during treatment can be accommodated efficiently, facilitating dynamic adjustments in response to real-time data.
The incorporation of technologies such as artificial intelligence (AI), in particular for image discrimination and target identification and geometry determination, can further improve the adaptability and responsiveness of the system, contributing to optimized therapeutic interventions.

As one should now appreciate, this invention has several advantages over known ultrasound therapy systems, including but not limited to US20090076390A1, U.S. Pat. No. 7,128,711B2, WO2013074661A3, and commercially available integrated imaging/therapy probes such as Sonatherm® and Ablatherm®.

Unlike systems that rely on mechanical repositioning of a therapy transducer or that integrate imaging and therapy functions into a single, shared transducer array with mode-switching, embodiments of the invention employ two separate, independently controlled two-dimensional matrix arrays: a high-density therapy array preferably having more than 1,000 individually controllable elements also preferably with element pitch less than 1.5× the wavelength, which enables precise volumetric beamforming of therapeutic ultrasound energy; and an imaging array configured for real-time three-dimensional ultrasound imaging, with electronic steering of the imaging focal position over a volumetric region.

In the integrated embodiments, these arrays may be housed in a common assembly and even lie in the same plane, but are electrically and functionally distinct, each with its own beamforming controller. This architecture allows simultaneous or near-simultaneous acquisition of volumetric imaging data and delivery of therapy without the compromises inherent in single-array mode-switching approaches such as those disclosed in US2024/0123456 (Stanford CMUT) or WO2013074661A3.

Furthermore, the invention incorporates a bidirectional guidance link between the imaging controller and therapy controller: In a first mode, the imaging subsystem determines target geometry and transmits 3D coordinates to the therapy subsystem for automated beam steering. In a second mode, the therapy subsystem transmits its instantaneous point-of-aim to the imaging subsystem for user-guided probe positioning.

Depending on the chosen embodiment, the invention also allows for multiple relative placements of the imaging and therapy arrays—including central, off-center, edge, or corner configurations—with software alignment compensation to maintain coincident therapeutic and imaging volumes. This contrasts with prior art systems, which generally restrict the imaging transducer to a fixed central position within a therapy transducer.

The system in various embodiments of the invention may also feature a continuous adaptive feedback loop: the imaging subsystem monitors the target in real time during therapy, halts treatment upon detecting motion beyond a threshold, and automatically reacquires target coordinates so that therapy can resume upon confirmation. This capability ensures both patient safety and targeting accuracy, and is absent from systems such as those that focus on robotic repositioning without real-time volumetric adaptive feedback.

In summary, the new system's advancements in transducer placement, imaging guidance, and adaptive treatment adjustments collectively result in a more user-friendly, accurate, and flexible approach to medical interventions, leading to improved patient care.

1. An ultrasound therapy system comprising:
a therapy transducer comprising an array of piezoelectric elements configured to transmit therapeutic ultrasound energy toward a target tissue;
a therapy controller operatively coupled to the therapy transducer and configured to independently energize the piezoelectric elements to cause the therapy transducer to focus the therapeutic ultrasound energy at a selectable focal position within a three-dimensional treatment region;
an imaging transducer configured to acquire ultrasound imaging data of the target tissue over a three-dimensional volume;
an imaging controller operatively coupled to the imaging transducer and configured to generate a three-dimensional ultrasound image of the target tissue from the imaging data and to cause display of a representation of the imaging data for viewing by a user; and
a communication link between the imaging controller and the therapy controller, the communication link being configured to transfer target location information from the imaging controller to the therapy controller,
wherein the therapy controller is configured to adjust the focal position of the therapeutic ultrasound energy within the three-dimensional treatment region based on the target location information.

2. The system of claim 1, wherein the therapy transducer and the imaging transducer are integrated within a common housing.

3. The system of claim 2, wherein the therapy transducer and the imaging transducer have piezoelectric elements disposed in a common plane but are energized and controlled by the separate respective controllers.

4. The system of claim 1, wherein the imaging transducer comprises a two-dimensional matrix array of piezoelectric elements and the imaging controller is configured to electronically steer an imaging focal position within the three-dimensional volume.

5. The system of claim 1, wherein the imaging transducer comprises a one-dimensional array of piezoelectric elements mechanically movable relative to the therapy transducer, and the imaging controller is configured to reconstruct the three-dimensional ultrasound image from a plurality of two-dimensional images acquired at different positions.

6. The system of claim 1, wherein
the imaging controller is configured to identify a boundary of the target tissue in the three-dimensional ultrasound image and determine three-dimensional coordinates of the target tissue, and
the therapy controller is configured to automatically focus the therapeutic ultrasound energy based on the three-dimensional coordinates.

7. The system of claim 1, wherein the therapy controller is configured to supply current focal position coordinates to the imaging controller for display in relation to the three-dimensional ultrasound image.

8. The system of claim 1, wherein element pitch of the therapy transducer is less than 1.5 times the wavelength of the transmitted ultrasound at a center frequency.

9. The system of claim 1, wherein the therapy controller is configured to dynamically change the focal position of the therapeutic ultrasound energy within the three-dimensional treatment region without mechanical movement of the therapy transducer.

10. The system of claim 1, wherein the imaging controller and therapy controller are further configured to operate in real time such that the therapy focal position is updated based on changes in the target tissue location detected in the three-dimensional ultrasound image.

11. The system of claim 1, wherein the system is further configured to halt transmission of therapeutic ultrasound energy upon detecting movement of the target tissue outside a predetermined range.

12. The system of claim 1, wherein the imaging transducer is located at a central position within the therapy transducer.

13. The system of claim 1, wherein the imaging transducer is located at an off-center position of the therapy transducer.

14. A method for performing ultrasound therapy guided by three-dimensional ultrasound images, comprising:
positioning a therapy transducer and an imaging transducer in proximity to a target tissue within a patient, said therapy and imaging transducers being included in a common housing;
acquiring ultrasound imaging data from the imaging transducer over a three-dimensional volume;
generating, by an imaging controller, a three-dimensional ultrasound image of the target tissue from the imaging data and displaying a representation of the imaging data for viewing by a user;
identifying a location of the target tissue within the three-dimensional ultrasound image;
transmitting the target location from the imaging controller to a therapy controller; and
focusing therapeutic ultrasound energy as a beam from the therapy transducer onto the target tissue based on the target location,
wherein the focusing is performed by electronically controlling timing and phasing of transmissions from elements of the therapy transducer without mechanical movement of the therapy transducer.

15. The method of claim 14, further comprising dynamically adjusting the focus of the therapeutic ultrasound energy within the three-dimensional volume in response to changes in the detected target tissue location.

16. The method of claim 14, wherein acquiring ultrasound imaging data comprises mechanically moving a one-dimensional array imaging transducer and reconstructing the three-dimensional image from multiple two-dimensional images acquired at different positions.

17. The method of claim 14, further comprising:
detecting a boundary of the target tissue within the three-dimensional ultrasound image; and
calculating three-dimensional coordinates defining the position, shape, and size of the target tissue.
focusing the therapeutic ultrasound energy comprises automatically adjusting a beamforming delay profile for the therapy transducer based on the three-dimensional coordinates.
computing a focal position of the therapeutic ultrasound beam;
transmitting focal position coordinates from the therapy controller to the imaging controller; and
displaying, within the three-dimensional ultrasound image, a representation of the focal position for user guidance prior to therapy activation.

18. The method of claim 14, further comprising automatically halting the transmission of therapeutic ultrasound energy upon detecting that the target tissue has moved outside a predetermined positional range.

19. The method of claim 14, wherein identifying the location of the target tissue comprises user marking of the target in the three-dimensional image, followed by automated tracking of the target tissue.
